(19) United States
(12) Patent Application Publication
(10) Pub. No.: US 2023/0284686 A1
(43) Pub. Date: Sep. 14, 2023

(54) AEROSOL PROVISION SYSTEM

(71) Applicant: Nicoventures Trading Limited, LONDON (GB)

(72) Inventors: Steve HUGHES, London (GB); Martyn STANIFORTH, London (GB); David Alan NELSON, London (GB)

(21) Appl. No.: 18/006,642

(22) PCT Filed: Jul. 9, 2021
(86) PCT No.: PCT/GB2021/051773

§ 371 Date: Jan. 24, 2023

(30) Foreign Application Priority Data
Jul. 24, 2020 (GB) 2011509.3

(51) Int. Cl. A24F 40/40; A24F 40/10; A24F 40/50

(52) U.S. Cl. CPC A24F 40/40; A24F 40/10; A24F 40/50; A24F 40/42

(57) ABSTRACT

An aerosol provision system including a vaporizer for generating a vapor from an aerosolizable material, and an electrode for receiving electrical power. The aerosol provision system also includes a resilient element, electrically connected to the vaporizer and the electrode, for transferring the electrical power between the electrode and the vaporizer. The aerosol provision system may include a cartridge and a control unit, wherein the electrode, the vaporizer, and the resilient element are located in the cartridge. The control unit may include a power supply for delivering electrical power to the electrode for powering the vaporizer.

### PRIORITY CLAIM

The present application is a National Phase entry of PCT Application No. PCT/GB2021/051773, filed Jul. 9, 2021, which claims priority from GB Application No. 2011509.3, filed Jul. 24, 2020, each of which hereby fully incorporated herein by reference.

### TECHNICAL FIELD

The present disclosure relates to aerosol provision systems such as, but not limited to, nicotine delivery systems (e.g. electronic cigarettes and the like).

### BACKGROUND

Electronic aerosol provision systems such as electronic cigarettes (e-cigarettes) generally contain an aerosol precursor material, such as a reservoir of a source liquid containing a formulation, typically but not necessarily including nicotine, or a solid material such a tobacco-based product, from which an aerosol is generated for inhalation by a user, for example through heat vaporization. Thus, an aerosol provision system will typically comprise a vaporizer, e.g. a heating element, arranged to vaporize a portion of precursor material to generate an aerosol in an aerosol generation region of an air channel through the aerosol provision system. As a user inhales on the device and electrical power is supplied to the heating element, air is drawn into the device through one or more inlet holes and along the air channel to the aerosol generation region, where the air mixes with the vaporized precursor material and forms a condensation aerosol. The air drawn through the aerosol generation region continues along the air channel to a mouthpiece opening, carrying some of the aerosol with it, and out through the mouthpiece opening for inhalation by the user.

It is common for aerosol provision systems to comprise a modular assembly, often having two main functional parts, namely a control unit and disposable/replaceable cartridge part. Typically the cartridge part will comprise the consumable aerosol precursor material and the vaporizer/heating element (atomizer), while the control unit part will comprise longer-life items, such as a power supply, such as a rechargeable battery, device control circuitry, activation sensors and user interface features. The control unit may also be referred to as a reusable part or battery section and the replaceable cartridge may also be referred to as a disposable part or cartomizer.

The control unit and cartridge are mechanically coupled together at an interface for use, for example using a screw thread, bayonet, latched or friction fit fixing. When the aerosol precursor material in a cartridge has been exhausted, or the user wishes to switch to a different cartridge having a different aerosol precursor material, the cartridge may be removed from the control unit and a replacement cartridge may be attached to the device in its place.

Electrical contacts/electrodes are provided on each of the control unit and cartridge for transferring power between the two components. In the case of each electrode on the cartridge, a lead is employed to transfer power from the electrode to the heating element in the cartridge.

A potential drawback in such cartridges is that the lead may become detached from the electrode during use, causing unwanted short-circuits and faulty operation of the cartridge. A potential further drawback for such cartridges, which typically contain liquid aerosol precursor (e-liquid) is the risk of leakage. An e-cigarette cartridge will typically have a mechanism, e.g. a capillary wick, for drawing liquid from a liquid reservoir to a heating element located in an air path/channel connecting from an air inlet to an aerosol outlet for the cartridge. Because there is a fluid transport path from the liquid reservoir into the open air channel through the cartridge, there is a corresponding risk of liquid leaking from the cartridge. Leakage is undesirable both from the perspective of the end user naturally not wanting to get the e-liquid on their hands or other items.

### SUMMARY

Various approaches are described herein which seek to help address or mitigate some of the issues discussed above. According to a first aspect of certain embodiments there is provided an aerosol provision system comprising: a vaporizer for generating a vapor from an aerosolizable material; an electrode for receiving power; and a resilient element, electrically connected to the vaporizer and the electrode, for transferring electrical power between the electrode and the vaporizer.

According to a second aspect of certain embodiments there is provided a cartridge for an aerosol provision system comprising the cartridge and a control unit, wherein the cartridge comprises: a vaporizer for generating a vapor from an aerosolizable material; an electrode for receiving power; and a resilient element, electrically connected to the vaporizer and the electrode, for transferring electrical power between the electrode and the vaporizer. According to a third aspect of certain embodiments there is provided an electrical power transmission system comprising: a first object for receiving electrical power; a second object; and a resilient element, electrically connected to the first object and the second object, wherein the resilient element is configured to transfer the electrical power between the first object and the second object, and wherein the second object is configured to be at least partly supported by the resilient element, and such that the resilient element is configured to be held in compression between the first object and the second object.

It will be appreciated that features and aspects of the disclosure described above in relation to the various aspects of the disclosure are equally applicable to, and may be combined with, embodiments of the disclosure according to other aspects of the disclosure as appropriate, and not just in the specific combinations described herein.

### BRIEF DESCRIPTION OF THE DRAWINGS

Embodiments of the disclosure will now be described, by way of example only, with reference to the accompanying drawings, in which:

FIG. 1 schematically represents an aerosol provision system comprising a cartridge and a control unit.

FIG. 2A schematically represents a cross sectional view of a cartridge, for use with the control unit from FIG. 1, in accordance with certain embodiments of the disclosure.

FIG. 2B shows a perspective view of portions of the cartridge shown in FIG. 2A, in accordance with certain embodiments of the disclosure.

FIG. 3 schematically shows a heating element, located on a surface of a porous member, for use in the cartridge shown in FIG. 2A in accordance with certain embodiments of the disclosure.

FIG. 4A schematically represents a cross sectional view of a portion of a cartridge, for use with the control unit from FIG. 1, in accordance with certain embodiments of the disclosure.

FIG. 4B schematically represents a perspective view of a portion of the cartridge from FIG. 4A, for use with the control unit from FIG. 1, in accordance with certain embodiments of the disclosure.

FIG. 5A schematically represents a cross sectional view of a portion of a cartridge, for use with the control unit from FIG. 1, in accordance with certain embodiments of the disclosure.

FIG. 5B schematically represents a perspective view of a portion of the cartridge from FIG. 5A, for use with the control unit from FIG. 1, in accordance with certain embodiments of the disclosure.

FIG. 6A schematically represents a cross sectional view of a cartridge, for use with the control unit from FIG. 1, in accordance with certain embodiments of the disclosure.

FIG. 6B schematically represents a perspective view of a portion of the cartridge from FIG. 6A, for use with the control unit from FIG. 1, in accordance with certain embodiments of the disclosure.

FIG. 6C schematically represents a perspective view of a portion of the cartridge from FIGS. 6A and 6B, for use with the control unit from FIG. 1, in accordance with certain embodiments of the disclosure.

FIG. 7 schematically represents a perspective view of a portion of a cartridge, for use with the control unit from FIG. 1, in accordance with certain embodiments of the disclosure.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional techniques for implementing such aspects and features.

The present disclosure relates to non-combustible aerosol provision systems, which may also be referred to as aerosol provision systems, such as e-cigarettes. According to the present disclosure, a “non-combustible” aerosol provision system is one where a constituent aerosolizable material of the aerosol provision system (or component thereof) is not combusted or burned in order to facilitate delivery to a user. Aerosolizable material, which also may be referred to herein as aerosol generating material or aerosol precursor material, is material that is capable of generating aerosol, for example when heated, irradiated or energized in any other way.

Throughout the following description the term “e-cigarette” or “electronic cigarette” may sometimes be used, but it will be appreciated this term may be used interchangeably with aerosol provision system/device and electronic aerosol provision system/device. An electronic cigarette may also known as a vaping device or electronic nicotine delivery system (END), although it is noted that the presence of nicotine in the aerosolizable material is not a requirement.

In some embodiments, the non-combustible aerosol provision system is a hybrid system to generate aerosol using a combination of aerosolizable materials, one or a plurality of which may be heated. In some embodiments, the hybrid system comprises a liquid or gel aerosolizable material and a solid aerosolizable material. The solid aerosolizable material may comprise, for example, tobacco or a non-tobacco product.

Typically, the non-combustible aerosol provision system may comprise a non-combustible aerosol provision device and an article for use with the non-combustible aerosol provision device. However, it is envisaged that articles which themselves comprise a means for powering an aerosol generating component may themselves form the non-combustible aerosol provision system.

In some embodiments, the article for use with the non-combustible aerosol provision device may comprise an aerosolizable material (or aerosol precursor material), an aerosol generating component (or vaporizer), an aerosol generating area, a mouthpiece, and/or an area for receiving aerosolizable material.

In some embodiments, the aerosol generating component is a vaporizer or heater capable of interacting with the aerosolizable material so as to release one or more volatiles from the aerosolizable material to form an aerosol. In some embodiments, the aerosol generating component is capable of generating an aerosol from the aerosolizable material without heating. For example, the aerosol generating component may be capable of generating an aerosol from the aerosolizable material without applying heat thereto, for example via one or more of vibrational, mechanical, pressurization or electrostatic means.

In some embodiments, the substance to be delivered may be an aerosolizable material which may comprise an active constituent, a carrier constituent and optionally one or more other functional constituents.

The active constituent may comprise one or more physiologically and/or olfactory active constituents which are included in the aerosolizable material in order to achieve a physiological and/or olfactory response in the user. The active constituent may for example be selected from nutraceuticals, nootropics, and psychoactives. The active constituent may be naturally occurring or synthetically obtained. The active constituent may comprise for example nicotine, caffeine, taurine, theine, a vitamin such as B6 or B12 or C, melatonin, a cannabinoid, or a constituent, derivative, or combinations thereof. The active constituent may comprise a constituent, derivative or extract of tobacco or of another botanical. In some embodiments, the active constituent is a physiologically active constituent and may be selected from nicotine, nicotine salts (e.g. nicotine ditartrate/nicotine bitartrate), nicotine-free tobacco substitutes, other alkaloids such as caffeine, or mixtures thereof.

In some embodiments, the active constituent is an olfactory active constituent and may be selected from a “flavor” and/or “flavorant” which, where local regulations permit, may be used to create a desired taste, aroma or other somatosensorial sensation in a product for adult consumers. In some instances such constituents may be referred to as flavors, flavorants, cooling agents, heating agents, and/or sweetening agents. They may include naturally occurring flavor materials, botanicals, extracts of botanicals, synthetically obtained materials, or combinations thereof (e.g., tobacco, Cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gasone or more of extracts (e.g., licorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder.

In some embodiments, the flavor comprises menthol, spearmint and/or peppermint. In some embodiments, the flavor comprises flavor components of cucumber, blueberry, citrus fruits and/or redberry. In some embodiments, the flavor comprises eugenol. In some embodiments, the flavor comprises flavor components extracted from tobacco. In some embodiments, the flavor may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucalyptol, WS-3.

The carrier constituent may comprise one or more constituents capable of forming an aerosol. In some embodiments, the carrier constituent may comprise one or more of glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

The one or more other functional constituents may comprise one or more of pH regulators, coloring agents, preservatives, binders, fillers, stabilizers, and/or antioxidants.

As noted above, aerosol provision systems (e-cigarettes) often comprise a modular assembly including both a reusable part (control unit) and a replaceable (disposable) cartridge part.

Devices conforming to this type of two-part modular configuration may generally be referred to as two-part devices. It is also common for electronic cigarettes to have a generally elongate shape. For the sake of providing a concrete example, certain embodiments of the disclosure described herein comprise this kind of generally elongate two-part device employing disposable cartridges. However, it will be appreciated the underlying principles described herein may equally be adopted for other electronic cigarette configurations, for example modular devices comprising more than two parts, as devices conforming to other overall shapes, for example based on so-called box-mod high performance devices that typically have a more boxy shape.

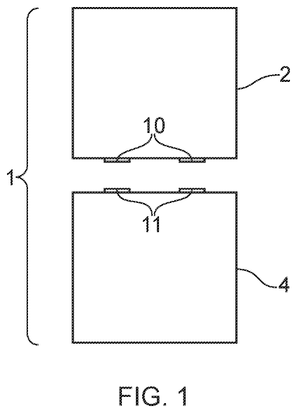

FIG. 1 is a schematic perspective view of an example aerosol provision system/device (e-cigarette) 1 in accordance with certain embodiments of the disclosure. Terms concerning the relative location of various aspects of the electronic cigarette (e.g. terms such as upper, lower, above, below, top, bottom etc.) are used herein with reference to the orientation of the electronic cigarette as shown in FIG. 1 (unless the context indicates otherwise). However, it will be appreciated this is purely for ease of explanation and is not intended to indicate there is any required orientation for the electronic cigarette in use.

The e-cigarette 1 comprises two main components, namely a cartridge 2 and a control unit 4. The control unit 4 and the cartridge 2 are coupled together when in use.

The cartridge 2 and control unit 4 are coupled by establishing a mechanical and electrical connection between them. The specific manner in which the mechanical and electrical connection is established is not of primary significance to the principles described herein and may be established in accordance with conventional techniques, for example based around a screw thread, bayonet, latched or friction-fit mechanical fixing with appropriately arranged electrical contacts/electrodes for establishing the electrical connection between the two parts as appropriate. For example, in the case of the cartridge 2 shown in FIG. 1, this cartridge 2 comprises a mouthpiece end 6 and an interface end 8. The cartridge 2 is coupled to the control unit 4 by a coupling arrangement (not shown in the Figures) at the interface end 8 of the cartridge 2 such to provide a releasable mechanical engagement between the cartridge and the control unit. An electrical connection is established between the control unit and the cartridge via a pair of electrical contacts/electrodes 10 on the bottom of the cartridge 2 and corresponding contact pins/electrodes 11 in the control unit 4. As noted above, the specific manner in which the electrical connection is established is not significant to the principles described herein. In accordance with a particular embodiment, the control unit 4 may comprise a cartridge receiving section that includes an interface arranged to cooperatively engage with the cartridge 2 so as to releasably couple the cartridge 2 to the control unit 4. In this way, electrical power from the control unit 4 may be delivered to the cartridge via the electrode 10 from the cartridge 2.

It will be appreciated the specific size and shape of the electronic cigarette and the material from which it is made is not of primary significance to the principles described herein and may be different in different implementations. That is to say, the principles described herein may equally be adopted for electronic cigarettes having different sizes, shapes and/or materials.

The control unit 4 may in accordance with certain embodiments of the disclosure be broadly conventional in terms of its functionality and general construction techniques. In some embodiments, the control unit may comprise a plastic outer housing including a receptacle wall that defines a receptacle for receiving the interface end 10 of the cartridge 2.

The control unit 4 further comprises a power supply, such as a battery for providing operating power for the electronic cigarette 1, control circuitry for controlling and monitoring the operation of the electronic cigarette, a user input button, and a charging port.

The battery in some embodiments may be rechargeable and may be of a conventional type, for example of the kind normally used in electronic cigarettes and other applications requiring provision of relatively high currents over relatively short periods. The power supply/battery may be recharged through the charging port, which may, for example, comprise a USB connector.

The input button may be considered an input device for detecting user input, e.g. to trigger aerosol generation, and the specific manner in which the button is implemented is not significant. For example, other forms of mechanical button or touch-sensitive button (e.g. based on capacitive or optical sensing techniques) may be used in other implementations, or there may be no button and the device may rely on a puff detector for triggering aerosol generation.

The control circuitry is suitably configured/programmed to control the operation of the electronic cigarette to provide conventional operating functions in line with the established techniques for controlling electronic cigarettes. The control circuitry (processor circuitry) may be considered to logically comprise various sub-units/circuitry elements associated with different aspects of the electronic cigarette's operation. For example, depending on the functionality provided in different implementations, the control circuitry may comprises power supply control circuitry for controlling the supply of power from the power supply/battery to the cartridge in response to user input, user programming circuitry for establishing configuration settings (e.g. user-defined power settings) in response to user input, as well as other functional units/circuitry associated functionality in accordance with the principles described herein and conventional operating aspects of electronic cigarettes. It will be appreciated the functionality of the control circuitry can be provided in various different ways, for example using one or more suitably programmed programmable computer(s) and/or one or more suitably configured application-specific integrated circuit(s)/circuitry/chip(s)/chipset(s) configured to provide the desired functionality.

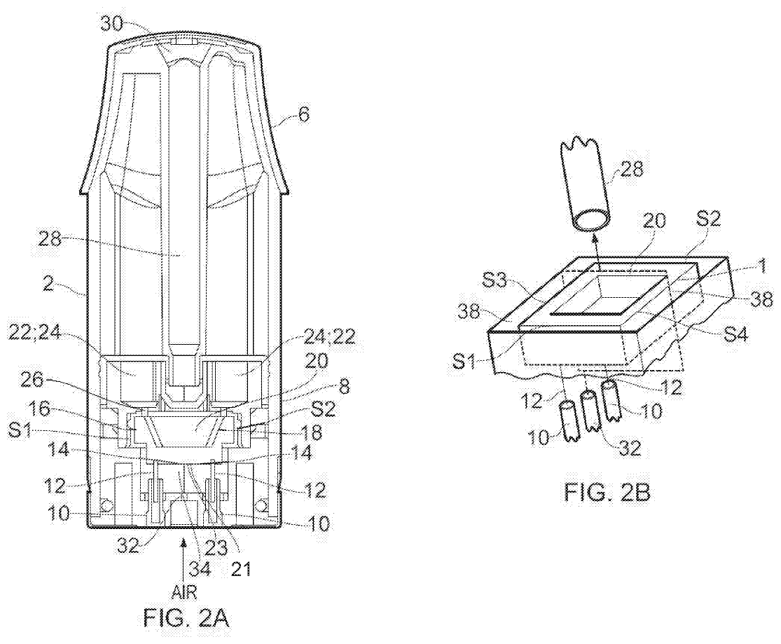

FIG. 2A schematically represents a cross sectional view of a cartridge, for use with the control unit from FIG. 1, in accordance with certain embodiments of the disclosure. In general terms, the cartridge comprises the electrodes 10, wherein each electrode 10 comprises an associated lead 12 which is operable to transfer power between the electrode 10 and a heating element 14. The cartridge 2 may further comprise a porous member 16 for use in holding a fluid to be atomized using the heating element 14. As shown in FIG. 2A, the porous member 16 may comprise a recess 18 defining a basin 20 for holding the fluid. In some embodiments, the porous member 16 may be a ceramic material, and may comprise silicone.

In the embodiment shown in FIG. 2A, the heating element 14 is located between the basin and each electrode 10. In terms of the structure of the heating element 14, in some embodiments the heating element 14 may be located on a surface 21 of the porous member 16.

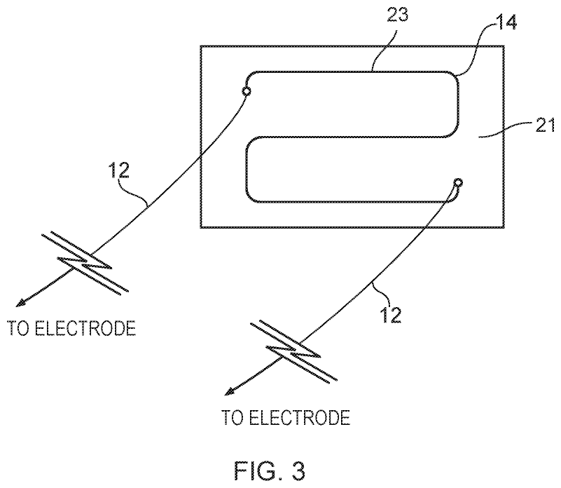

In the case of the embodiments shown in FIGS. 2A and 3, the surface 21 is located on an opposite side of the porous member to that of the basin 20.

To improve the transfer of heat from the heating element to the porous member 16, in some embodiments the heating element 14 may comprise a metal wire or some other conductive material, which may form a tortuous path 23 on the surface 21 of the porous member 16. In that arrangement, a first end of the heating element may be connected to one of the two leads 12, and a second end opposite the first end of the heating element connected to the other of the two leads 12. In terms of the exact shape of the heating element 14, it will be appreciated that the heating element 14 in such embodiments may take any required shape on the surface of the porous member 16 for efficiently vaporizing the aerosolizable material/fluid in the porous member 16. In that respect, and in accordance with some particular embodiments, the heating element/vaporizer 14 may define a spiral pattern; a raster pattern; or a zig-zag pattern on the surface of the porous member 16.

Located towards the mouthpiece end 6 of the cartridge is a chamber 22 acting as a primary reservoir 24 for storing fluid to be aerosolized. The chamber 22 is connected to the basin 20 via at least one opening 26 for topping up the level of fluid in the basin 20, which acts a secondary reservoir.

Extending through the center of the chamber 22 is an outlet channel 28 for receiving aerosol generated from fluid emanating from the porous member 16. The outlet channel 28 extends from the porous member up towards a mouthpiece 30 located at the mouthpiece end 6 of the cartridge, for allowing a user to inhale the aerosol which is generated.

The cartridge comprises an air channel 32 extending through the cartridge for delivering air to the heating element 14. In the embodiment shown in FIG. 2A, the air channel 32 is located between the electrodes 10. Upon connection of the cartridge 2 with the control unit 4, the electronic cigarette 1 would be provided with a further air channel located in the cartridge 2 and/or the control unit 4 which is in fluid communication with the air channel 32, and which is configured to allow ambient air to be passed therethrough and into air channel 32.

The heating element 14 is located in an aerosol generation region 34 from the cartridge 2, and the outlet channel 28 and the air channel 30 are connected to the aerosol generation region 34.

In normal use, the cartridge 2 is coupled to the control unit 4 and the control unit activated to supply power to the cartridge 2 via the electrodes 10;11. Power then passes through the connection leads 12 to the heating element 14.

The function of the porous member 16 is to act as a capillary wick for drawing fluid from the basin 20 to the heating element 14. Accordingly, fluid which is wicked towards the heating element 14 through the porous member 16 is vaporized by the heat generated from the heating element 14. The generated vapor emanates from the surface 21 where it mixes with the air from the air channel 32 in the aerosol generation region 34 to form an aerosol. Fluid which is vaporized from the porous member 16 is replaced by more fluid drawn from the chamber 22 via the at least one opening 26.

Air enters the air channel 32 as a result of the user inhaling on the mouthpiece 30 of the cartridge 2. This inhalation causes air to be drawn through whichever further air channel aligns with the air channel 32 of the cartridge. The incoming air mixes with aerosol generated from the heating element 14 to form a condensation aerosol at the underside of the porous member 16 in the aerosol generation region 34. The formed aerosol then passes from the underside of the porous member 16, past a gap 38 located on two sides S3;S4 of the porous member as shown in FIG. 2B (the sides S3;S4 being perpendicular to the sides S1;S2 shown in FIG. 2A), and then up through the outlet channel 28 to the mouthpiece 30.

The above therefore describes a cartridge 2 for an aerosol provision system, wherein the cartridge 2 comprises a heating element/vaporizer 14 located in an aerosol generation region 34 from the cartridge 2, and is for heating/vaporizing fluid from a reservoir 20;24 to generate aerosol in the aerosol generation region 34, wherein the cartridge 2 further comprises an air channel 32 extending through the cartridge 2 for delivering air to the heating element/vaporizer 14.

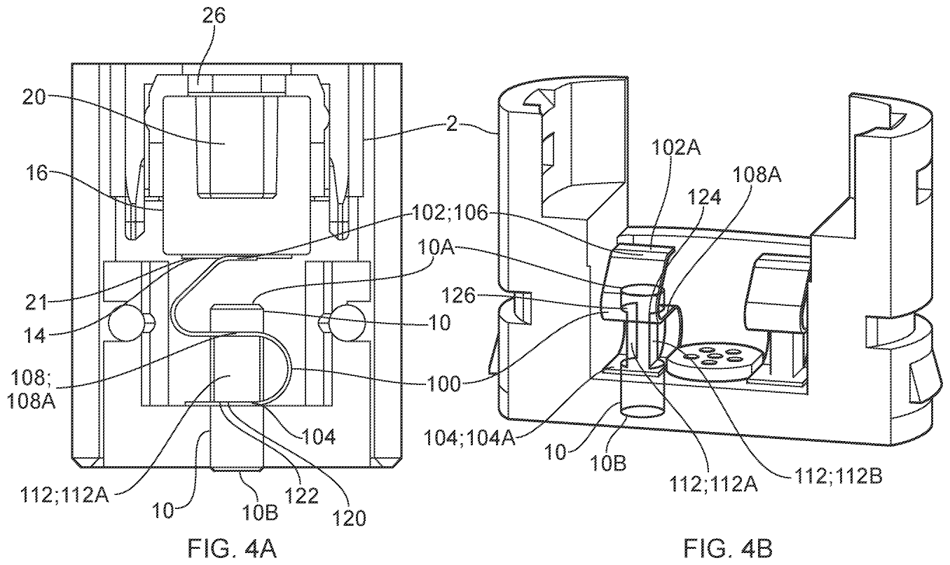

With reference to FIGS. 4A-7, there are schematically shown modified cartridges 2 for use with the control unit 4 shown in FIG. 1 to form an aerosol provision system 1 in accordance with certain embodiments of the disclosure. The cartridge 2, or portions thereof, shown in FIGS. 4A-7 are based on the construction of cartridge 2 shown in FIGS. 1-3, and comprise similar components as set out by the reference numerals that are common to both sets of Figures. For instance, the cartridge 2 comprises the at least one electrode 10, the heating element/vaporizer 14, and the porous member 16.

A principal modification to the cartridge 2 shown in FIGS. 4A-7 over the cartridge shown in FIGS. 2A-3 is the introduction of a resilient member 100 to replace all or part of the connection lead 12. In this respect, the connection leads 12 may become detached from the electrode 10 during use, causing unwanted short-circuits and faulty operation of the cartridge 2. A potential further drawback is that with such connection leads 12, which are shown in FIGS. 2A-2B as embedded in the electrode 10, fluid/vapor may ingress in the gap between the connection lead 12 and the electrode 10, which may impact on the efficiency in any electrical power transmitted between the connection lead 12 and the electrode 10, e.g. as a result of corrosion forming in this gap.

From the foregoing therefore, and as will be described, the disclosure from FIGS. 4A-7 effectively provide an aerosol provision system 1 comprising a vaporizer 14 for generating a vapor from an aerosolizable material; an electrode 10 for receiving electrical power; and a resilient element 100, electrically connected to the vaporizer 14 and the electrode 10, for transferring the electrical power between the electrode 10 and the vaporizer 14. As will be described, via the introduction of this resilient element 100, this may notionally alleviate the aforementioned disadvantages caused by use of the connection lead(s) 12.

Mindful of the above, and starting with the disclosure from FIGS. 4A-4B, the resilient element 100 may be provided with a first portion 102 proximal the vaporizer 14 and a second portion 104 proximal the electrode 10. In accordance with such embodiments, such as that shown in FIGS. 4A-4B, the second portion 104 may effectively be in contact with the electrode 10, with the first portion 102 in contact with the vaporizer 14. That being said, in accordance with some embodiments, there may be provided at least one electrically conductive bridging member situated between the first portion 102 and the vaporizer 14, and/or situated between the second portion 104 and the electrode 10.

In accordance with the above embodiments, the first portion 102 may define a cantilevered portion 106 on which the vaporizer 14 and/or the porous member 16 (where present) is supported. Put differently, the resilient element 100 in accordance with such embodiments may be configured to support (at least partially or fully) the vaporizer 14 and/or the porous member 16, as is shown n FIGS. 4A-7. In that way, and in accordance with some embodiments, the resilient element 100 may by configured to provide a biasing force and/or compression force on the vaporizer 14, which in some particular embodiments (such as that shown in FIGS. 4A-7) may extend in a direction away from the electrode 10.

From the above therefore, it can be seen that resilient nature of the resilient element 100 may cause it to be biased into engagement with the vaporizer 14, such that the resilient element 100 is held in compression in use. In that respect, and in accordance with a particular embodiment, the resilient element 100 may therefore comprise a leaf spring, which is held in compression in use.

In accordance with some particular embodiments, such as that shown in FIGS. 4A-4B, the resilient element 100 may define an S shape. In that way, and in accordance with some embodiments, the resilient element 100 may comprise a third portion 108, which is located between the first portion 102 and the second portion 104, and which is in contact with the electrode 10.

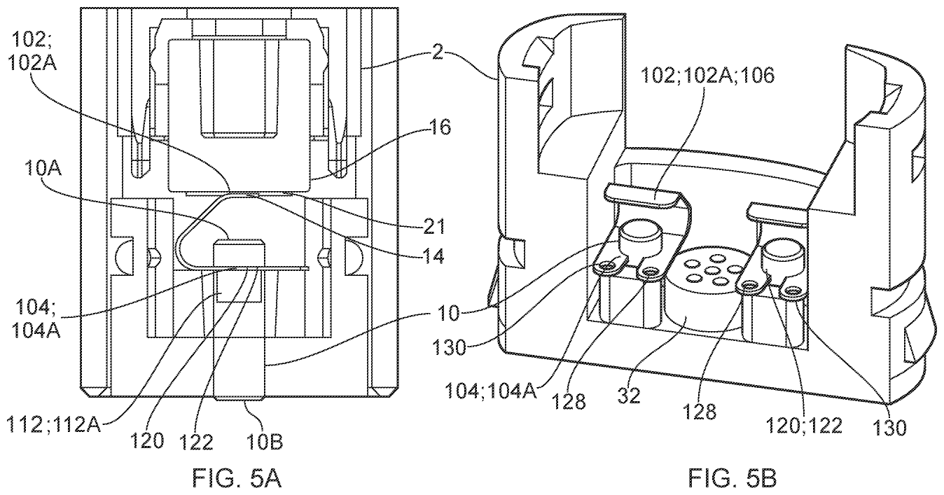

During the operation of the resilient element 100, the electrode 10 may be configured to rest, either directly or indirectly, on the resilient element 100 in at least one location from the resilient element 100, such to appropriately support the resilient element 100 relative to the electrode 10. In accordance with some embodiments, the at least one least one location may comprise the second portion 104 (such as in the embodiments shown in FIGS. 5A-5B) and/or comprise the third portion 108 (as in the embodiment shown in FIGS. 4A-4B).

To provide additional securement of the resilient member 100 to the electrode 10, in accordance with some embodiments, the electrode 10 may comprise at least one recess 112 for accommodating the resilient element 100. Such a recess is shown in the embodiment of FIGS. 4A-4B. In accordance with such embodiments, and other embodiments, the at least one recess 112 of the electrode 10 may comprise a plurality of recesses 112A;112B, such as in particular a first recess 112A located on a first side of the electrode 10, and a second recess 112B located on a second side of the electrode 10 which is opposite the first side (as shown in the embodiment of FIGS. 4A-4B). With the provision of this recess(es) 112 in the electrode 10, this may allow the resilient element 100 to engage inside the recess(es) 112 for thus restricting any unwanted movement/slip of the electrode 10 relative to the resilient element 100.

Staying with the geometry of the electrode, in at least some embodiments (such as those shown in FIGS. 4A-7), the electrode 10 may extend between a first end 10A of the electrode 10 and a second end 10B of the electrode 10, wherein the first end 10A of the electrode 10 is located more proximal to the vaporizer 14 than the second end 10B of the electrode is located to the vaporizer 14, and wherein the first end 10A in accordance with some particular embodiments thereof may be located opposite the second end 10B (for instance in the case of the electrode being cylindrical). In accordance with such geometry, this may allow for a convenient spacing and positioning of the electrode 10 relative to the vaporizer 14 and the resilient element 100.

In accordance with some particular embodiments of the above geometry, a particularly compact embodiment which optimizes space may comprise the first end 10A of the electrode 10 located between the first portion 102 and the third portion 108 of the resilient element 100. Such an embodiment is shown in FIGS. 4A-4B. In accordance with such embodiments, and notionally others such as that shown in FIGS. 5A-5B, the first and second ends 10A;10B of the electrode may be located on opposite sides of the second portion 104.

Where any recess(es) 112 is present in the electrode 10, it will be appreciated that any such recess(es) may facilitate the resilient element 100 to be held in compression by the electrode 10, noting such compression facilities a function of the resilient element 100 to provide support to the vaporizer 14 and the porous member 16 (where present) in use, as can be seen from the embodiment of FIGS. 4A-4B.

To further provide adequate support by the resilient element 100 with respect to its neighboring electrode 10 and/or the vaporizer 14, and to provide an increased surface area for the transferring of electrical power between the electrode 10 and the vaporizer 14, in accordance with some embodiments, any combination of the first portion 102; the second portion 104; and the third portion 108 (where any of these portions are present) may respectively comprise a flat plate (first flat plate 102A; second flat plate 104A; and third plate 108A) to improve the contact/support by the resilient element 100 in that respective portion. In that respect as well, any combination of these flat plates 102A;104A;108A may in accordance with some particular embodiments be parallel to each other, to ensure a more stable arrangement for the resilient element 100, and in which loading forces therein may be better distributed.

Focusing further on the interconnection between the resilient element 100 and the electrode 10, and with reference to the embodiment shown in FIGS. 4A-4B, in accordance with some embodiments, the resilient element 100 may comprise at least one recess in which a portion of the electrode 10 is accommodated. Each such recess may therefore serve to assist in securing the resilient element 100 relative to the electrode 10. In some specific embodiments thereof, the second portion 104 of the resilient element 100 may comprise a primary recess 120 in which a primary portion 122 of the electrode 10 is accommodated; and/or the third portion 108 may comprise a secondary recess 124 in which a secondary portion 126 of the electrode 10 is accommodated.

Moving away from the S shaped resilient element shown in the embodiment shown in FIGS. 4A-4B, and turning to that shown in FIG. 5A-5B, in accordance with some embodiments, the resilient element 100 may be configured to act as a circlip which extends around the electrode for gripping the electrode 10. In that respect, and in accordance with some embodiments, the resilient element 100 may comprise resilient feet 130 which extend around the electrode 10 for gripping the electrode 10. In accordance with some particular embodiments thereof, such as that shown in FIGS. 5A-5B, the resilient feet 130 may form part of the second portion 104 (and/or part of the second flat plate 104A).

As noted above, one of the primary functions of the resilient element 100 is to transfer electrical power between the electrode 10 and the vaporizer 14. That being the case, it is envisaged that the resilient element 100 may comprise an electrically conductive material (such as, but not limited to, a metal) for transferring the electrical power received by the electrode 10 to the vaporizer 14. Inferably, and as will be appreciated, this may be achieved by the electrically conductive material being deposited onto the resilient element (e.g. as a coating extending around the surface of the resilient element, or a strip of electrically conductive material being located/deposited thereon), and/or the resilient element being made of the electrically conductive material.

In accordance with some embodiments, to further secure the resilient means 100 in its location with respect to the cartridge 2, the resilient means 100 may be provided with at least one fastening point 128, such as a hole or a fastener, for fastening the resilient means 100 to another object (such as the cartridge 2). In accordance with some embodiments, such as that shown in FIGS. 5A-5B, the fastening point 128 may be located on the second portion 104 and/or the second flat plate 104A. As required in accordance with some particular embodiments, the at least one fastening point 128 may comprises a plurality of fastening points to provide further securement of the resilient means 100. In the very particular embodiment where the resilient element 100 is configured to act as a circlip which extends around the electrode 10 for gripping the electrode 10, each fastening point 128 may also act as the point where the circlip may be gripped for locating the circlip around the electrode 10, as shown in the embodiment of FIGS. 5A-5B (where each fastening point 128 comprises a hole extending through each resilient foot portion 130).

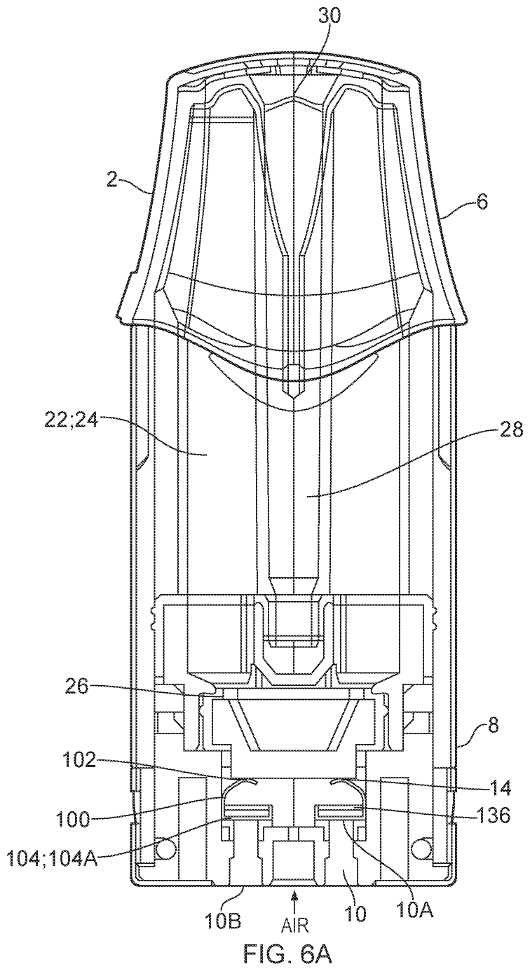

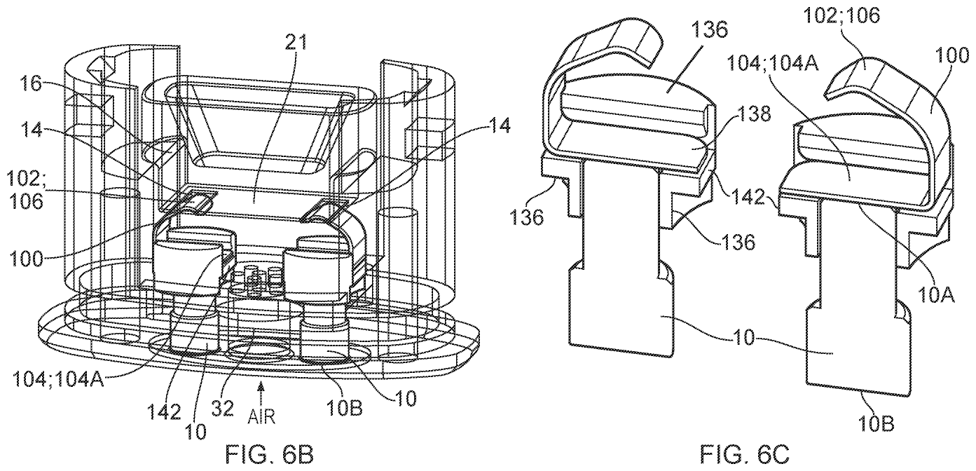

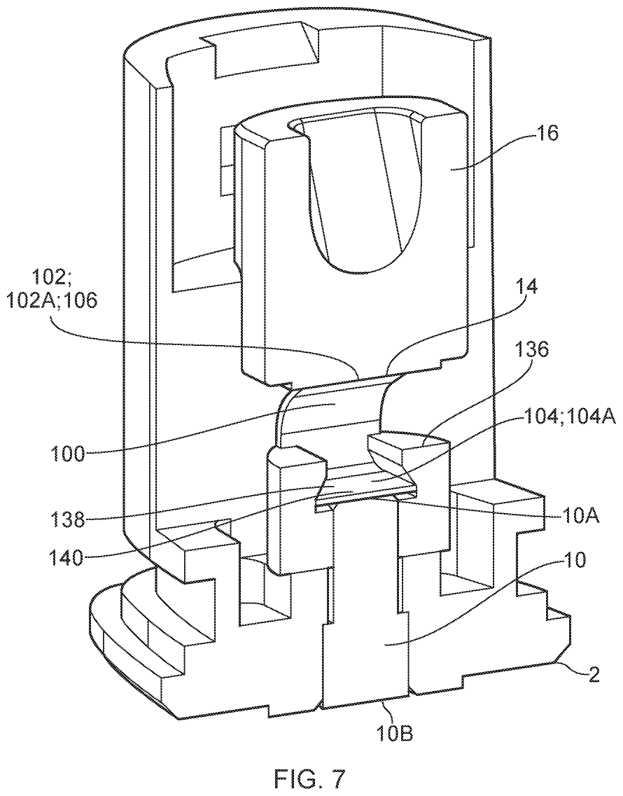

Staying with the securing of the resilient means 100, and turning to FIGS. 6A-7, in accordance with some embodiments, the electrode 10 may comprise a cover member 136 defining a recess 138, in which the second portion 104 of the resilient element 100 is configured to be received, for restricting the removal of the resilient element 100 from the electrode 10. A primary purpose of the cover member 136, as noted above, is to accommodate the resilient element for restricting the removal of the resilient element 100 from the electrode 10. In that respect therefore, it will be appreciated that the recess 138 could take any required shape to achieve this functionality. In accordance with some particular embodiments, the recess 138 might define a slot, which may define a substantially rectangular cross-section. To provide further securement of the resilient element 100, the cover member 136 in accordance with some embodiments may be configured to grip the second portion 104 of the resilient element 100 for keeping the second portion 106 in contact with the electrode 10. With reference to some very particular embodiments, such as those shown in FIGS. 6A-7, the cover member 136 may be configured to grip the second portion 106 of the resilient element 100 for keeping the second portion 106 in contact with the first end 10A of the electrode 10. In accordance with some particular embodiments for further restricting any upward separation/lifting of the resilient element relative to the electrode 10, the recess 136 may define a tapered or trapezoidal cross section 140 for restricting the detachment of the second portion 106 from the electrode 10, and/or for restricting any movement of the second portion 106 in a direction towards the vaporizer 14, in use. Such an arrangement is illustrated in the embodiment of FIG. 7.

In accordance with some embodiments where the cover member 136 is employed, in some particular embodiments thereof, the cover member 136 may comprises keyed surface 142 for preventing the cover member 136 from rotating relative to the electrode 10. The shape of this keyed surface 142 may appreciably take any required shape to achieve this effect. In that respect therefore, and in accordance with some embodiments, such as that shown in FIGS. 6A-6C, the keyed surface 142 may be configured to engage against a portion of the cartridge 2, such as a wall of the air channel 32. The exact shape of the keyed surface 142 may therefore vary, as required, so long as the surface can assist with preventing the cover member 136 from rotating relative to the electrode 10. In that respect therefore, as possible non-limiting examples, the keyed surface 142 might comprise a flat surface (as shown in the embodiment of FIGS. 6A-6C), a castellated surface, or comprise a recess and/or projection for engaging with a corresponding projection and/or recess from the object in which the keyed surface is configured to be located in use (e.g. the cartridge 2).

Where the cover member 136 is present, a further function thereof may be to restrict the ingress of vapor/moisture around the electrode 10, noting such vapor/moisture may ultimately contribute to undesirable corrosion of the electrode 10. With that in mind, the cover member 136 in accordance with some embodiments may be configured to extend around the first end 10A of the electrode 10. In this respect as well, the cover member 136 may be notionally made of a material which is relatively electrically insulting, and which is relatively impervious to moisture. Accordingly, in accordance with some embodiments, the cover member may be made of plastic. In a very particular (non-limiting) embodiment), it has been found that the cover member 136 being made of Poly-Cyclohexylenedimethylene Terephthalate Glycol (PCTG) provides the cover member 136 with particularly favorable properties in the above respect.

With respect to the resilient member 100 described herein and as illustrated in the embodiments from FIGS. 4A-7, it is envisaged (as noted previously) that this resilient member 100 may be used with some of the other previously described features of the aerosol provision system 1 described with reference to FIGS. 1-3, such as but not limited the porous member 16, the vaporizer 14, and any of the other features from the cartridge 2 or control unit 4 shown in FIGS. 1-3 which collectively form the aerosol provision systems 1 described herein. Accordingly, there has been described an aerosol provision system comprising: a vaporizer for generating a vapor from an aerosolizable material; an electrode for receiving electrical power; and a resilient element, electrically connected to the vaporizer and the electrode, for transferring the electrical power between power the electrode and the vaporizer.

There has also been described a cartridge for an aerosol provision system comprising the cartridge and a control unit, wherein the cartridge comprises: a vaporizer for generating a vapor from an aerosolizable material; an electrode for receiving electrical power from the control unit; and a resilient element, electrically connected to the vaporizer and the electrode, for transferring the electrical power between the electrode and the vaporizer.

There has also been described an aerosol provision system 1 comprising a vaporizer 14 for generating a vapor from an aerosolizable material, and an electrode 10 for receiving electrical power. The aerosol provision system 1 also comprises a resilient element 100, electrically connected to the vaporizer 14 and the electrode 10, for transferring the electrical power between the electrode 10 and the vaporizer 14. The aerosol provision system 1 may comprise a cartridge 2 and a control unit 4, wherein the electrode 10, the vaporizer 14, and the resilient element 100 are located in the cartridge 2. The control unit 4 may comprise a power supply for delivering electrical power to the electrode 10 for powering the vaporizer 14.

For the sake of completeness however, it is to be noted that the resilient element 100 described herein need not be expressly used in an aerosol provision system 1 which comprises a cartridge 2 and the control unit 4. Accordingly, the resilient element 100 may be notionally used in any aerosol provision system 1 which is configured to generate a vapor from an aerosolizable material.

In this respect as well, and at an even broader level, it is envisaged that the resilient element 100 described herein may have wider applications for transferring power from a first object to another object which is configured to receive the power from the first object, and which is also configured to be at least partly supported by the resilient element, such that the resilient element is configured to act as a load-bearing element for the second object. In other words, the resilient element 100 need not expressly be for use with an electrode 10 (the first object) and the vaporizer 14 (the second object) specifically. That being the case, described herein may also be an electrical power transmission system comprising: a first object for receiving electrical power; a second object; and a resilient element, electrically connected to the first object and the second object, wherein the resilient element is configured to transfer the electrical power between the first object and the second object, and wherein the second object is configured to be supported (such as at least partly, or fully) by the resilient element, and such that the resilient element is configured to be held in compression between the first object and the second object.

Although not necessarily, in accordance with some of these embodiments, the first object may comprise the electrode 10 and/or the second object may comprise the vaporizer 14 for generating a vapor from an aerosolizable material.

Where the resilient element 100 is configured to be used in such an electrical power transmission system, it will be appreciated that the resilient element 100 may comprise any of the features and/or functionality as described herein, such as (but not limited to) the resilient element 100 comprising the cover member 136 (for locating/extending around the first object), and/or the cantilevered portion 106, the resilient feet 130 for extending around the first object, and/or the resilient portion 100 defining an S shape.

Also in respect of the resilient element 100 described herein, it will be appreciated that there may be provided one or more resilient elements 100, as required, depending on how many electrodes 10 (first objects) there are. Accordingly, although the description has been principally described with reference to the operation of a single resilient element 100, it will be appreciated (as noted in FIGS. 4A-7) that more than one resilient element 100 may in practice be employed, as required, such as there being one resilient element for each provided electrode 10. In that respect as well, and purely for the avoidance of any doubt, where more than one resilient element 100 is provided, the plurality of resilient elements 100 may all electrically connect to a single vaporizer 14 (second object) and/or electrically connect to a separate vaporizer 14 (second object) for each electrode 100, depending on the particular application of the resilient element 100. In that respect, and with reference to the embodiment shown in FIGS. 4A-6C, there may in accordance with some particular embodiments be provided an aerosol provision system 1 comprising the vaporizer 14 for generating a vapor from an aerosolizable material; a plurality of electrodes 10 for receiving electrical power; and a plurality of resilient elements 100, wherein each resilient element 100 is electrically connected to the vaporizer 14 and a respective one of the electrodes 10, for transferring the electrical power between the respective one of the electrodes 10 and the vaporizer 14.

In order to address various issues and advance the art, this disclosure shows by way of illustration various embodiments in which the claimed invention(s) may be practiced. The advantages and features of the disclosure are of a representative sample of embodiments only, and are not exhaustive and/or exclusive. They are presented only to assist in understanding and to teach the claimed invention(s). It is to be understood that advantages, embodiments, examples, functions, features, structures, and/or other aspects of the disclosure are not to be considered limitations on the disclosure as defined by the claims or limitations on equivalents to the claims, and that other embodiments may be utilized and modifications may be made without departing from the scope of the claims. Various embodiments may suitably comprise, consist of, or consist essentially of, various combinations of the disclosed elements, components, features, parts, steps, means, etc. other than those specifically described herein, and it will thus be appreciated that features of the dependent claims may be combined with features of the independent claims in combinations other than those explicitly set out in the claims. The disclosure may include other inventions not presently claimed, but which may be claimed in future.

For instance, although the present disclosure has been described with reference to a “liquid” or “fluid” in the cartridge/aerosol provision system, it will be appreciated that this liquid or fluid may be replaced with any aerosolizable material. Equally, where an aerosolizable material is used, it will be appreciated that in some embodiments this aerosolizable material may comprise a liquid or fluid.

Furthermore, whilst the present disclosure has been described with reference to a heater/heating element being present in the cartridge/aerosol provision system, it will be appreciated that in accordance with some embodiments this heating element may be replaced with a vaporizer or some other aerosol generating component. Equally, such an aerosol generating component in accordance with some embodiments may in particular comprise a heater or heating element.

The present disclosure also provides the embodiments as set out in the following numbered clauses:
1. An aerosol provision system comprising:
   a vaporizer for generating a vapor from an aerosolizable material;
   an electrode for receiving electrical power; and
   a resilient element, electrically connected to the vaporizer and the electrode, for transferring the electrical power between the electrode and the vaporizer.
2. An aerosol provision system according to any preceding clause, wherein the resilient element comprises a first portion proximal the vaporizer and a second portion proximal the electrode.
3. An aerosol provision system according to clause 2, wherein the first portion is in contact with the vaporizer.
4. An aerosol provision system according to clause 3, wherein the first portion comprises a first flat plate.
5. An aerosol provision system according to any of clauses 2-4, wherein the second portion comprises a second flat plate.
6. An aerosol provision system according clause 5, when further dependent on clause 4, wherein the first flat plate is parallel to the second flat plate.
7. An aerosol provision system according to any of clauses 2-6, wherein the second portion is in contact with the electrode.
8. An aerosol provision system according to any of clauses 2-7, wherein the second portion comprises a primary recess in which a primary portion of the electrode is accommodated.
9. An aerosol provision system according to any of clauses 2-8, wherein the resilient element comprises a third portion, located between the first portion and the second portion, which is in contact with the electrode.
10. An aerosol provision system according to clause 9, wherein the third portion comprises a third flat plate.
11. An aerosol provision system according to clause 10, when further dependent on clause 4 or 5, wherein the third flat plate is parallel to at least one of the first flat plate and the second flat plate.
12. An aerosol provision system according to any of clauses 9-11, wherein the third portion comprises a secondary recess in which a secondary portion of the electrode is accommodated.
13. An aerosol provision system according to any of clauses 1-12, wherein the electrode is configured to rest on the resilient element in at least one location from the resilient element.
14. An aerosol provision system according to clause 13, when further dependent on clause 2, wherein the at least one location comprises the second portion.
15. An aerosol provision system according to clause 13 or 14, when further dependent on clause 9, wherein the at least one location comprises the third portion.
16. An aerosol provision system according to any of clauses 1-15, wherein the electrode comprises at least one recess for accommodating the resilient element.
17. An aerosol provision system according to clause 16, wherein the at least one recess of the electrode comprises a plurality of recesses.
18. An aerosol provision system according to any of clauses 1-17, wherein the electrode extends between a first end of the electrode and a second end of the electrode, wherein the first end of the electrode is located more proximal to the vaporizer than the second end of the electrode is located to the vaporizer.
19. An aerosol provision system according to clause 18, when further dependent on clause 16, wherein the at least one recess is located between the first end of the electrode and the second end of the electrode.
20. An aerosol provision system according to clause 18 or 19, when further dependent on clause 9, wherein the first end of the electrode is located between the first portion and the third portion of the resilient element.
21. An aerosol provision system according to any of clauses 1-20, wherein the resilient element is configured to provide at least one of a biasing force and/or a compression force on the vaporizer.
22 An aerosol provision system according to clause 21, wherein the at least one of a biasing force and a compression force extends in a direction away from the electrode.
23. An aerosol provision system according to any of clauses 1-22, wherein the resilient element is biased into engagement with the vaporizer.
24. An aerosol provision system according to any of clauses 1-23, wherein the resilient element comprises a leaf spring.
25. An aerosol provision system according to any of clauses 1-24, wherein the resilient element defines an S shape.
26. An aerosol provision system according to any of clauses 1-25, wherein the resilient element is configured to act as a circlip which extends around the electrode for gripping the electrode.
27. An aerosol provision system according to any of clauses 1-26, wherein the resilient element comprises resilient feet which extend around the electrode for gripping the electrode.
28. An aerosol provision system according to any of clauses 1-27, wherein the resilient element is held in compression by the electrode.
29. An aerosol provision system according to any of clauses 1-28, when further dependent on clause 2, wherein the electrode comprises a cover member defining a recess, in which the second portion of the resilient element is configured to be received, for restricting the removal of the resilient element from the electrode.
30. An aerosol provision system according to clause 29, when further dependent on clause 18, wherein the cover member extends around the first end of the electrode.
31. An aerosol provision system according to clause 29 or 30, wherein the cover member is configured to grip the second portion of the resilient element for keeping the second portion in contact with the electrode.
32. An aerosol provision system according to clause 31, when further dependent on clause 18, wherein the cover member is configured to grip the second portion of the resilient element for keeping the second portion in contact with the first end of the electrode.
33. An aerosol provision system according to any of clauses 29-32, wherein the cover member comprises a keyed surface for preventing the cover member from rotating relative to the electrode.
34. An aerosol provision system according to any of clauses 29-33, wherein the cover member is made of plastic.
35. An aerosol provision system according to any of clauses 29-34, wherein the cover member is made of Poly-Cyclohexylenedimethylene Terephthalate Glycol (PCTG).
36. An aerosol provision system according to any of clauses 1-35, wherein the resilient element comprises an electrically conductive material for transferring the electrical power received by the electrode to the vaporizer.
37. An aerosol provision system according to clause 36, wherein the electrically conductive material is deposited onto the resilient element.
38. An aerosol provision system according to clause 36, wherein the resilient element is made of the electrically conductive material.
39. An aerosol provision system according to any of clauses 1-38, further comprising a porous member for use in holding aerosolizable material to be vaporized using the vaporizer.
40. An aerosol provision system according to clause 39, wherein the resilient element at least partly supports the porous member, such that the resilient element is configured to be held in compression between the porous member and the electrode.
41. An aerosol provision system according to clause 39 or 40, when further dependent on clause 2, wherein the first portion defines a cantilevered portion on which the porous member is supported.
42. An aerosol provision system according to any of clauses 1-41, when further dependent on clause 2, wherein the first portion defines a cantilevered portion on which the vaporizer is supported.
43. An aerosol provision system according to any of clauses 1-42, wherein the resilient element at least partly supports the vaporizer, such that the resilient element is configured to be held in compression between the vaporizer and the electrode.
44. An aerosol provision system according to any of clauses 1-43, wherein the vaporizer comprises a heating element.
45. An aerosol provision system according to any of clauses 1-44, further comprising a reservoir for aerosolizable material, wherein the vaporizer is configured to receive the aerosolizable material from the reservoir.
46. An aerosol provision system according to any of clauses 1-45, further comprising a cartridge and a control unit, wherein the electrode, the vaporizer, and the resilient element are located in the cartridge, wherein the control unit comprises a cartridge receiving section that includes an interface arranged to cooperatively engage with the cartridge so as to releasably couple the cartridge to the control unit, wherein the control unit further comprises a power supply for delivering electrical power to the electrode for powering the vaporizer.
47. A cartridge for an aerosol provision system comprising the cartridge and a control unit, wherein the cartridge comprises:
   a vaporizer for generating a vapor from an aerosolizable material;
   an electrode for receiving electrical power from the control unit; and
   a resilient element, electrically connected to the vaporizer and the electrode, for transferring the electrical power between the electrode and the vaporizer.

1-46. (canceled)

47. An aerosol provision system comprising:
a vaporizer for generating a vapor from an aerosolizable material, wherein the vaporizer comprises a heating element;
a porous member for use in holding aerosolizable material to be vaporized using the vaporizer;
an electrode for receiving electrical power; and
a resilient element, electrically connected to the vaporizer and the electrode, for transferring the electrical power between the electrode and the vaporizer, wherein the resilient element is biased into engagement with the vaporizer, and wherein the resilient element comprises an electrically conductive material for transferring the electrical power received by the electrode to the vaporizer,
wherein the resilient element comprises a first portion proximal the vaporizer and a second portion proximal the electrode, the first portion is in contact with the vaporizer, the first portion comprises a first flat plate, and the first portion defines a cantilevered portion on which the vaporizer is supported, and
wherein the resilient element is configured to provide at least one of a biasing force or a compression force on the vaporizer, and the at least one of the biasing force or the compression force extends in a direction away from the electrode.

48. The aerosol provision system according to claim 47, wherein the second portion comprises a second flat plate.

49. The aerosol provision system according to claim 47, wherein the electrode comprises at least one recess for accommodating the resilient element.

50. The aerosol provision system according to claim 47, wherein the electrode extends between a first end of the electrode and a second end of the electrode, wherein the first end of the electrode is located more proximal to the vaporizer than the second end of the electrode is located to the vaporizer.

51. The aerosol provision system according to claim 47, wherein the resilient element is held in compression by the electrode.

52. The aerosol provision system according to claim 47, wherein the electrically conductive material is deposited onto the resilient element.

53. The aerosol provision system according to claim 47, wherein the resilient element is made of the electrically conductive material.

54. The aerosol provision system according to claim 47, wherein the resilient element at least partly supports the vaporizer, such that the resilient element is configured to be held in compression between the vaporizer and the electrode.

55. The aerosol provision system according to claim 47, further comprising a reservoir for the aerosolizable material, wherein the vaporizer is configured to receive the aerosolizable material from the reservoir.

56. The aerosol provision system according to claim 47, further comprising a cartridge and a control unit,
wherein the electrode, the vaporizer, and the resilient element are located in the cartridge, and
wherein the control unit comprises a cartridge receiving section that includes an interface arranged to cooperatively engage with the cartridge so as to releasably couple the cartridge to the control unit, and the control unit further comprises a power supply for delivering electrical power to the electrode for powering the vaporizer.

57. A cartridge for an aerosol provision system comprising the cartridge and a control unit, wherein the cartridge comprises:
a vaporizer for generating a vapor from an aerosolizable material, wherein the vaporizer comprises a heating element;
a porous member for use in holding aerosolizable material to be vaporized using the vaporizer;
an electrode for receiving electrical power from the control unit; and
a resilient element, electrically connected to the vaporizer and the electrode, for transferring the electrical power between the electrode and the vaporizer, wherein the resilient element is biased into engagement with the vaporizer, and wherein the resilient element comprises an electrically conductive material for transferring the electrical power received by the electrode to the vaporizer,
wherein the resilient element comprises a first portion proximal the vaporizer and a second portion proximal the electrode, the first portion is in contact with the vaporizer, the first portion comprises a first flat plate, and the first portion defines a cantilevered portion on which the vaporizer is supported, and
wherein the resilient element is configured to provide at least one of a biasing force or a compression force on the vaporizer, the at least one of the biasing force or the compression force extends in a direction away from the electrode.
